(19) United States
(12) Patent Application Publication
(10) Pub. No.: US 2025/0176825 A1
(43) Pub. Date: Jun. 5, 2025

(54) INFORMATION PROCESSING APPARATUS AND INFORMATION PROCESSING METHOD

(71) Applicant: CANON KABUSHIKI KAISHA, Tokyo (JP)

(72) Inventors: Kohtaro UMEZAWA, Tokyo (JP); Yoshihiko IWASE, Kanagawa (JP)

(21) Appl. No.: 18/935,715

(22) Filed: Nov. 4, 2024

(30) Foreign Application Priority Data
Nov. 30, 2023 (JP) 2023-203333

(51) Int. Cl. A61B 3/107; A61B 3/12; A61B 3/14; G06F 3/14; G06T 7/00; G06T 7/50

(52) U.S. Cl. CPC A61B 3/107; A61B 3/12; A61B 3/14; G06F 3/14; G06T 7/0012; G06T 7/50; G06T 2207/30041

(57) ABSTRACT

Provided is an information processing apparatus including: a tomographic image acquisition unit configured to acquire a tomographic image of a fundus of an eye to be inspected; a shape abnormality information acquisition unit configured to acquire shape abnormality information; a tomographic abnormality information acquisition unit configured to acquire tomographic abnormality information; and a display control unit configured to perform control of displaying at least part of the shape abnormality information and at least part of the tomographic abnormality information such that the at least part of the shape abnormality information and the at least part of the tomographic abnormality information are distinguishable from each other.

### BACKGROUND OF THE INVENTION

### Field of the Invention

The present invention relates to an information processing apparatus and an information processing method.

### Description of the Related Art

A fundus tomographic image pickup apparatus such as an optical coherence tomography (OCT) apparatus allows three-dimensional observation of a state inside of a retinal layer. The tomographic image pickup apparatus such as an OCT image pickup apparatus has been gathering attention in recent years because of its effectiveness in performing disease diagnosis more accurately.

Further, there is also a technology of detecting an abnormal region that may be related to a disease or the like from the tomographic image of the fundus. This technology has also been gathering attention in recent years because of its effectiveness in supporting the disease diagnosis.

In Japanese Patent Application Laid-Open No. 2016-2380, there is disclosed a technology of displaying information on a shape of a retinal layer, such as a layer thickness or a curvature in the tomographic image, in superimposition with a fundus image. In Japanese Patent Application Laid-Open No. 2016-2380, a two-dimensional map (hereinafter also referred to as “shape map”) in which the information on the shape of the retinal layer has been projected onto a surface along the fundus (hereinafter also referred to as “fundus parallel surface”) is acquired, and further, a two-dimensional map in which an abnormal region in the shape map has been imaged is acquired.

Further, in Japanese Patent Application Laid-Open No. 2022-160184, there is disclosed a technology of displaying an abnormal region in a tomographic image in superimposition with a shape map.

The abnormal region detected from the information on the shape of the retinal layer as disclosed in Japanese Patent Application Laid-Open No. 2016-2380 and the abnormal region detected based on the tomographic image as disclosed in Japanese Patent Application Laid-Open No. 2022-160184 are abnormal regions detected through processes different from each other. Accordingly, there is a demand for a measure for allowing a relationship between those abnormal regions to be easily grasped.

### SUMMARY OF THE INVENTION

The present invention has been made in order to solve the above-mentioned problem.

That is, according to one aspect of the present invention, there is provided an information processing apparatus including: a tomographic image acquisition unit configured to acquire a tomographic image of a fundus of an eye to be inspected; a shape abnormality information acquisition unit configured to acquire, based on the tomographic image, shape abnormality information which is information about a part having an abnormal shape in a retinal layer of the eye to be inspected; a tomographic abnormality information acquisition unit configured to acquire, based on a tomographic abnormality region which is an abnormal region in the tomographic image, tomographic abnormality information which is information about a part having the tomographic abnormality region in the retinal layer of the eye to be inspected; and a display control unit configured to perform control of displaying at least part of the shape abnormality information and at least part of the tomographic abnormality information such that the at least part of the shape abnormality information and the at least part of the tomographic abnormality information are distinguishable from each other.

Further features of the present invention will become apparent from the following description of exemplary embodiments with reference to the attached drawings.

### BRIEF DESCRIPTION OF THE DRAWINGS

FIG. 1 is a block diagram for illustrating a functional configuration of an information processing apparatus according to a first embodiment.

FIG. 2 is a block diagram for illustrating a hardware configuration of the information processing apparatus according to the first embodiment.

FIG. 3 is a flow chart for illustrating an example of a procedure of an information processing method performed by the information processing apparatus according to the first embodiment.

FIG. 4 is a view for illustrating an example of a display screen in the first embodiment.

FIG. 5 is a view for illustrating an example of a display screen in the first embodiment.

FIG. 6 is a view for illustrating an example of a display screen in the first embodiment.

### DESCRIPTION OF THE EMBODIMENTS

Exemplary embodiments of the present invention are described in detail below with reference to the attached drawings. The embodiments described below do not limit the present invention set forth in the appended claims. A plurality of features are described in the embodiments, but the present invention does not necessarily require all of those plurality of features, and a plurality of features may be combined as appropriate.

The present invention is preferably applicable to a workstation connected to an OCT image pickup apparatus, a workstation for performing image analysis, or a radiogram interpretation terminal viewer. Examples of the OCT image pickup apparatus include a time domain OCT apparatus and a spectral domain OCT apparatus.

### FIRST EMBODIMENT

In a first embodiment, an information processing apparatus for displaying a tomographic abnormality region, a tomographic abnormality map, a shape abnormality map, and a shape abnormality back-projection region in a distinguishable manner is described. The tomographic abnormality region in the first embodiment is an abnormal region of a specific tomographic image in OCT volume data obtained by photographing a fundus of an eye to be inspected by an OCT image pickup apparatus. Further, the tomographic abnormality map in the first embodiment is a two-dimensional map obtained by projecting the tomographic abnormality regions in all of the tomographic images onto a fundus parallel surface. Further, the shape abnormality map in the first embodiment is a map indicating an abnormal region in a shape map which is a two-dimensional map obtained by projecting information on a shape of a specific retinal layer in each tomographic image onto the fundus parallel surface for all of the tomographic images. Further, the shape abnormality back-projection region in the first embodiment is a region obtained by back-projecting the shape abnormality map onto the specific tomographic image.

Here, the “back-projection” refers to, in the first embodiment, processing of obtaining a region on a tomographic image corresponding to a predetermined region on the shape map or on the shape abnormality map.

### Functional Configuration of Information Processing Apparatus

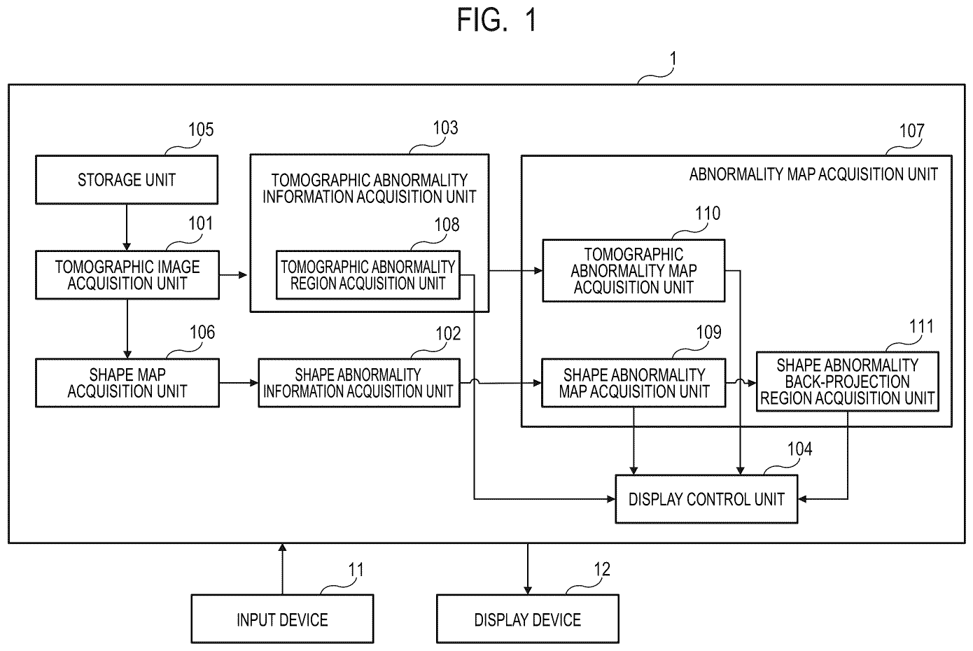

FIG. 1 is a block diagram for illustrating a functional configuration of the information processing apparatus according to the first embodiment. An information processing apparatus 1 includes a tomographic image acquisition unit 101, a shape abnormality information acquisition unit 102, a tomographic abnormality information acquisition unit 103, a display control unit 104, and a storage unit 105. In the first embodiment, the information processing apparatus 1 further includes a shape map acquisition unit 106 and an abnormality map acquisition unit 107. Further, the tomographic abnormality information acquisition unit 103 includes a tomographic abnormality region acquisition unit 108, and the abnormality map acquisition unit 107 includes a shape abnormality map acquisition unit 109, a tomographic abnormality map acquisition unit 110, and a shape abnormality back-projection region acquisition unit 111. The information processing apparatus 1 is connected to an input device 11 and a display device 12, and may be further connected to an external apparatus (not shown) such as the OCT image pickup apparatus via a network.

### Hardware Configuration of Information Processing Apparatus

The information processing apparatus 1 is formed of a computer including a processor, a memory, a storage, and the like. In this case, each step of processing and each function included in the information processing apparatus 1 are implemented by loading a program stored in the storage onto the memory and causing the processor to execute the program. Examples of the function referred to here include the shape abnormality information acquisition unit 102, the tomographic abnormality information acquisition unit 103, the shape map acquisition unit 106, the shape abnormality map acquisition unit 109, the tomographic abnormality region acquisition unit 108, the tomographic abnormality map acquisition unit 110, the shape abnormality back-projection region acquisition unit 111, and the display control unit 104. However, the present invention is not limited thereto, and, for example, the whole or some of the functions described above may be implemented by a specifically designed processor (such as an application specific integrated circuit (ASIC)) or a field programmable gate array (FPGA). As another example, part of the arithmetic processing may be executed by a processor such as a graphics processing unit (GPU) or a digital signal processor (DSP). Further, the information processing apparatus 1 may be formed of a single piece of hardware or a plurality of pieces of hardware. For example, cloud computing or distributed computing may be used so that a plurality of computers cooperate with each other to implement the functions and the processing of the information processing apparatus 1.

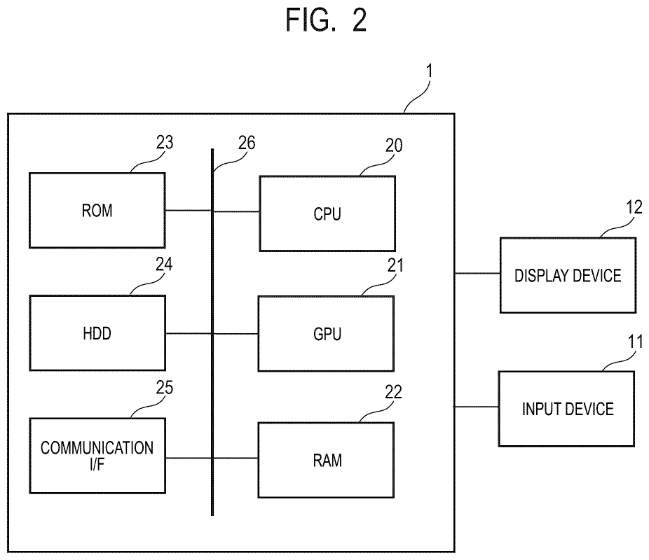

FIG. 2 is a block diagram for illustrating an example of a hardware configuration of the information processing apparatus 1. In this example, the information processing apparatus 1 includes a central processing unit (CPU) 20, a GPU 21, a random access memory (RAM) 22, a read only memory (ROM) 23, a hard disk drive (HDD) 24, and a communication interface (I/F) 25, and those components are connected to each other via a system bus 26.

The CPU 20 and the GPU 21 are processors for reading out programs stored in the ROM 23 and the HDD 24 onto the RAM 22 and executing the programs, to thereby perform arithmetic processing, control of each unit of the information processing apparatus 1, or the like.

The RAM 22 is a volatile storage medium, and functions as a work memory when the CPU 20 and GPU 21 execute the program. The ROM 23 is a non-volatile storage medium, and stores firmware required for the operation of the information processing apparatus 1, or the like. The HDD 24 is a non-volatile storage medium, and stores, for example, a tomographic image and information on an abnormality in a retinal layer acquired from the tomographic image in the first embodiment.

The communication I/F 25 is a communication device based on a standard such as Wi-Fi (trademark), Ethernet (trademark), or Bluetooth (trademark). The communication I/F 25 is used for communication to/from the OCT image pickup apparatus, other computers, and the like.

The display device 12 is a device from which the information processing apparatus 1 outputs information to the outside, and is typically a user interface for presenting the information to a user. Examples of the display device 12 include a display accompanying the information processing apparatus 1, and a mobile terminal of a medical staff member communicating via an external server. The display device 12 displays display information generated by the display control unit 104.

The input device 11 is a device for inputting information to the information processing apparatus 1, and is typically a user interface for allowing the user to operate the information processing apparatus 1. Examples of the input device 11 include a keyboard, buttons, a mouse, and a touch panel.

The above-mentioned configuration of the information processing apparatus 1 is merely an example, and can be changed as appropriate. Examples of a processor that can be mounted in the information processing apparatus 1 include an ASIC, and an FPGA in addition to the above-mentioned CPU 21. In addition, a plurality of those processors may be provided, or a plurality of processors may perform processing in a distributed manner. Further, the function of storing information such as image data in the HDD 24 may be included in another data server instead of in the information processing apparatus 1. Further, the HDD 24 may be a storage medium such as an optical disc, a magneto-optical disk, or a solid state drive (SSD).

### Operation of Information Processing Apparatus

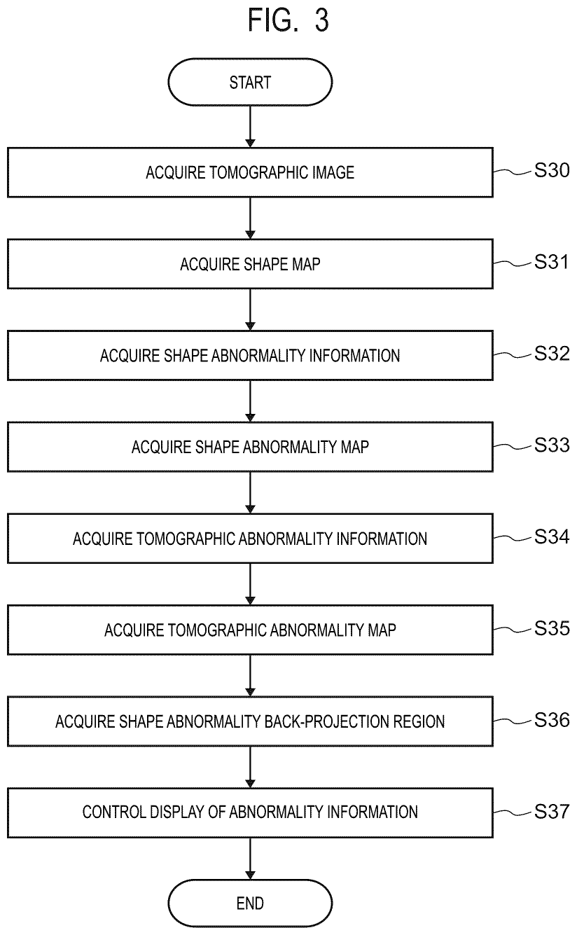

Next, with reference to a flow chart of FIG. 3, an information processing method in the information processing apparatus 1 according to the first embodiment is described.

### Step S30: Acquisition of Tomographic Image

In a tomographic image acquisition step of Step S30, the tomographic image acquisition unit 101 acquires a tomographic image of a fundus of an eye to be inspected stored in the storage unit 105. In the first embodiment, a tomographic image picked up as follows is used as the tomographic image. That is, while one-dimensional scan (hereinafter also referred to as “A-scan”) is performed from the OCT image pickup apparatus to the retina in a depth direction in an optical axis direction, continuous scan (hereinafter also referred to as “B-scan”) is performed by laterally shifting the optical axis. The tomographic image acquisition unit 101 acquires a two-dimensional tomographic image (hereinafter also referred to as “B-scan image”) picked up as described above.

The direction in which the optical axis is shifted in order to acquire the B-scan image is not particularly limited, and the B-scan image may be acquired with the optical axis being shifted in any direction as long as the tomographic image of the retinal layer can be obtained. Further, three-dimensional volume data (hereinafter referred to as “OCT volume data”) itself acquired from the OCT image pickup apparatus may be used as the tomographic image. In this case, the OCT volume data is obtained by performing continuous scan of the B-scan while shifting in a direction perpendicular to the B-scan image.

Now, an example in which the OCT volume data of the fundus of the eye to be inspected picked up by the OCT image pickup apparatus is acquired and used as the tomographic image is described. The OCT volume data acquired by the tomographic image acquisition unit 101 is stored in the storage unit 105.

### Step S31: Acquisition of Shape Map

In a shape map acquisition step of Step S31, the shape map acquisition unit 106 acquires a shape map of a first predetermined retinal layer visualized in the OCT volume data acquired in Step S30. Here, the shape map acquired by the shape map acquisition unit 106 is a two-dimensional map obtained by projecting information on a shape of the first predetermined retinal layer obtained from all of the tomographic images onto the fundus parallel surface.

Then, the shape map acquisition unit 106 transmits the acquired shape map to the shape abnormality information acquisition unit 102. Further, the shape map acquisition unit 106 transmits the information on the first predetermined retinal layer to the shape abnormality information acquisition unit 102 as layer information of the shape map (information indicating which layer the shape map focuses on).

Here, the retinal layer is known to include, from the cornea side, an inner limiting membrane (ILM), a retinal nerve fiber layer (NFL), a ganglion cell layer (GCL), an inner plexiform layer (IPL), an inner nuclear layer (INL), an outer plexiform layer (OPL), an outer nuclear layer (ONL), an outer limiting membrane (OLM), photoreceptor outer segments (POS), a retinal pigment epithelium (RPE), and a choroid.

In the first embodiment, the information on the shape is a layer thickness of the first predetermined retinal layer, and is acquired by the following method. First, segmentation of the retinal layer is executed for each tomographic image in the OCT volume data. Then, out of regions obtained as a result of the segmentation, at least one or more predetermined layers (for example, ILM to RPE) are focused on, and a one-dimensional distribution of the thickness (information on the shape) in each of the tomographic images is acquired. Then, the one-dimensional distribution of the thickness (information on the shape) acquired in each tomographic image is projected onto the fundus parallel surface (mapped on a corresponding line). Subsequently, the one-dimensional distributions of the thicknesses (pieces of information on the shape) projected in the respective tomographic images are all arranged (integrated) so that a two-dimensional shape map (layer thickness map) is acquired. There may also be employed a configuration in which, instead of acquiring one shape map, a plurality of different shape maps for a plurality of layers (for example, ILM to GCL and ILM to INL) are acquired as analysis targets. Further, the information on the shape may be information other than the layer thickness, for example, a curvature of a specific retinal layer (at a boundary between a layer and a layer). Further, there may also be employed a configuration in which a plurality of pieces of information on the shape, such as a layer thickness and a curvature, are each treated as the information on the shape (that is, a shape map of each piece of information is regarded as the analysis target).

How to acquire the shape map is not limited to a method of projecting the information on the shape acquired in each tomographic image described above onto the fundus parallel surface for all of the tomographic images and integrating the results. For example, the shape map may be acquired by, after performing three-dimensional segmentation from the OCT volume data and acquiring the information on the shape, projecting the information on the shape onto the fundus parallel surface.

Next, as the layer information (first predetermined retinal layer) of the shape map, out of the regions obtained as the result of the segmentation at the time of acquiring the shape map, information on at least one or more predetermined layers (for example, ILM to RPE) focused as the analysis target is acquired. In the first embodiment, the user inputs this information through use of the input device 11 such as the mouse or the keyboard. Then, this information is transmitted to the shape abnormality information acquisition unit 102. There may also be employed a configuration in which a previously-defined range stored in advance in the storage unit 105 is used as the first predetermined retinal layer.

### Step S32: Acquisition of Shape Abnormality Information

In a shape abnormality information acquisition step of Step S32, the shape abnormality information acquisition unit 102 acquires, based on the tomographic image, shape abnormality information which is information about a part having an abnormal shape in the retinal layer of the eye to be inspected. In the first embodiment, the shape abnormality information acquisition unit 102 acquires, by subjecting the shape map of the eye to be inspected acquired in Step S31 to analysis processing, information about a region having an abnormal shape that may have been caused based on a disease in the shape map. Then, the shape abnormality information and layer information of the shape abnormality information are transmitted to the shape abnormality map acquisition unit 109 included in the abnormality map acquisition unit 107.

In the first embodiment, the shape abnormality information is acquired by the following method. First, a deep learning model trained so as to output, through use of any shape map as input, a shape map of a healthy eye (hereinafter also referred to as “healthy eye shape map”) is acquired from the storage unit 105. This deep learning model has been trained through use of only shape maps obtained from tomographic images of a fundus of a health eye, that is, tomographic images having no abnormality, and is a trained model for outputting a shape map restored based on the shape map corresponding to the input.

Next, the shape map acquired from the tomographic image of the fundus of the eye to be inspected is input to the above-mentioned deep learning model to cause the deep learning model to output the healthy eye shape map. In the first embodiment, a healthy eye layer thickness map which is a healthy eye shape map about the layer thickness of the retinal layer is acquired. Then, the shape abnormality information is acquired based on a difference between the shape map of the eye to be inspected (that is, a measured value) and the output healthy eye shape map (that is, an estimated normal value). For example, a region in which a difference value between both values (that is, the measured value and the estimated normal value) at a corresponding position exceeds a range set in advance is determined as a region having a shape abnormality.

In the description above, the shape abnormality information is acquired based on the difference value between the shape map of the eye to be inspected and the healthy eye shape map, but the shape abnormality information may be acquired based on values other than the difference value as long as a method of quantifying a deviation from the healthy eye shape map is employed. For example, the shape abnormality information may be acquired based on an absolute value of the difference between both values. Further, the shape abnormality information may be acquired based on a ratio between both values. For example, a region in which the value of the ratio exceeds a predetermined range may be determined as the abnormal region.

The method of acquiring the shape abnormality information is not limited to the above-mentioned method. For example, a segmentation model of deep learning that has been trained so as to output, through use of any shape map as input, a normal region and an abnormal region in the map in a discriminated manner may be acquired from the storage unit 105 and used. Examples of such a trained model include an abnormality detection model that has been trained through use of a shape map having no abnormal region as training data. When this segmentation model is applied to the shape map of the eye to be inspected, the shape abnormality information can be acquired. Further, the shape abnormality information may be acquired based on, after a reference database in which shape maps of a plurality of healthy eyes are averaged is created in advance, deviation from the reference database of the shape map of the eye to be inspected. That is, there may also be employed a configuration in which a healthy eye reference shape map acquired from the reference database is used in place of the healthy eye shape map in the processing described above.

In the first embodiment, the layer information (first predetermined retinal layer) of the shape map is acquired as the layer information of the shape abnormality information.

When a plurality of shape maps are acquired in Step S31, the shape abnormality information may be acquired from each of the shape maps.

The abnormality map acquisition unit 107 has a function of acquiring an abnormality map representing the shape abnormality information described above and tomographic abnormality information to be described later on at least one of a surface along the fundus of the eye to be inspected and a surface in a direction intersecting perpendicularly with the fundus of the eye to be inspected. In the first embodiment, an example in which the abnormality map acquisition unit 107 includes the shape abnormality map acquisition unit 109, the tomographic abnormality map acquisition unit 110, and the shape abnormality back-projection region acquisition unit 111 is described.

### Step S33: Acquisition of Shape Abnormality Map

In a shape abnormality map acquisition step of Step S33, the shape abnormality map acquisition unit 109 acquires a shape abnormality map which is a map representing the shape abnormality information acquired in Step S32 on the surface along the fundus of the eye to be inspected. Then, the shape abnormality map acquisition unit 109 transmits the shape abnormality map and layer information of the shape abnormality map to the display control unit 104.

Specifically, the shape abnormality map acquisition unit 109 generates a shape abnormality map in which, in the shape abnormality information acquired in Step S32, a value of 1 is given to a region determined as having a shape abnormality and a value of 0 is given to other regions. The threshold value for determining the shape abnormality may be determined in advance and stored in the storage unit 105, or may be input and determined by the user through the input device 11.

Further, the shape abnormality map may be a map representing the degree of the abnormality (abnormality degree) not by a binary value but by a continuous value. For example, a function for calculating an abnormality degree of from 0 to 1 by using the absolute value of the difference between both values for each pixel as input may be defined, and the shape abnormality map may be generated through use of this function. There may be used, as the function, such an increasing function that is 0 when the input value is small and comes closer to 1 when the input value is large. Further, a ratio between the absolute value of the difference between both values and the healthy eye shape map may be calculated as the abnormality degree of each pixel, and the distribution of the abnormality degrees may be used as the shape abnormality map.

In the first embodiment, the layer information (first predetermined retinal layer) of the shape map is acquired as the layer information of the shape abnormality map (information indicating which layer the shape abnormality map focuses on).

When a plurality of pieces of shape abnormality information are acquired in Step S32, the shape abnormality map may be generated for each of the pieces of shape abnormality information.

In the first embodiment, the method of acquiring the shape abnormality map based on the shape map has been described, but the present invention is not limited thereto. The shape abnormality map acquisition unit 109 may acquire the shape abnormality map by any other method as long as a map representing, on the fundus parallel surface, a part having abnormality of the shape can be obtained from the shape abnormality information.

### Step S34: Acquisition of Tomographic Abnormality Information

In a tomographic abnormality information acquisition step of Step S34, the tomographic abnormality information acquisition unit 103 acquires tomographic abnormality information which is information about a part including an abnormal region in a retinal layer of the eye to be inspected, based on a tomographic abnormality region which is an abnormal region in the tomographic image. In the first embodiment, the tomographic abnormality region acquisition unit 108 acquires, with respect to the tomographic image in the OCT volume data acquired in Step S30, an abnormal region (tomographic abnormality region) that may have been caused based on a disease in the image, and transmits the abnormal region to the display control unit 104. Further, the tomographic abnormality region acquisition unit 108 acquires layer information (information identifying a layer in which the tomographic abnormality region is present) of the tomographic abnormality region, and transmits the layer information together with the position of the tomographic image to the display control unit 104. In this case, the user may input and designate the tomographic image to be selected in the OCT volume data through use of the input device 11 such as the mouse or the keyboard. In the first embodiment, an example in which the user inputs (clicks), on a shape abnormality map display window 402 to be described later, the position of the tomographic image desired to be displayed (hereinafter referred to as “display tomographic image”) through use of the mouse so as to determine the position is described.

In the first embodiment, the tomographic abnormality region acquisition unit 108 acquires the tomographic abnormality region by the following method. First, a segmentation model of deep learning that has been trained so as to output, through use of a tomographic image as input, an abnormality degree (degree of similarity to the abnormal region) of each pixel in the image is acquired from the storage unit 105. Examples of the segmentation model of the deep learning include an abnormality detection model that has been trained through use of the tomographic image having no abnormality as training data.

Subsequently, the above-mentioned segmentation model is applied to the tomographic image of the eye to be inspected, and the output (abnormality degree image) of the segmentation model is acquired. Then, this image is subjected to binarization processing through use of a threshold value set in advance so that the tomographic abnormality region is acquired in a form of a binary image (tomographic abnormality region image) representing whether each pixel of the tomographic image is normal (0) or abnormal (1). The tomographic abnormality region may be acquired in a form of expressing the degree of similarity to the abnormal region as a continuous value, like the abnormality degree image output by the segmentation model described above.

The tomographic abnormality region acquisition unit 108 may acquire the tomographic abnormality region for each of the plurality of tomographic images (B-scan images) forming the OCT volume data.

Further, the tomographic abnormality region acquisition unit 108 may perform three-dimensional segmentation on the OCT volume data to acquire the tomographic abnormality region as three-dimensional information.

The tomographic abnormality information acquisition unit 103 acquires the tomographic abnormality information obtained by integrating the tomographic abnormality region obtained for the tomographic image and information about the retinal layer having the tomographic abnormality region.

In the first embodiment, the information about the retinal layer having the tomographic abnormality region is acquired by the following method. That is, the tomographic abnormality region of the tomographic image of the eye to be inspected is compared with a segmentation result of the retinal layer of the tomographic image so that information indicating which layer in the tomographic image has the tomographic abnormality region (for example, ILM to RPE) is acquired.

The tomographic abnormality information acquisition unit 103 may acquire, for example, the following information in place of the information about the retinal layer having the tomographic abnormality region (for example, ILM to RPE). That is, the tomographic abnormality information acquisition unit 103 may acquire information on whether or not the tomographic abnormality region is included in the range represented by the layer information (for example, ILM to GCL) of the shape map described above (that is, in the first predetermined retinal layer). That is, the tomographic abnormality information acquisition unit 103 is not always required to acquire the information about all of the retinal layers having the tomographic abnormality region, and may acquire information about only some of the retinal layers having the tomographic abnormality region.

### Step S35: Acquisition of Tomographic Abnormality Map

In a tomographic abnormality map acquisition step of Step S35, the tomographic abnormality map acquisition unit 110 acquires a tomographic abnormality map which is a map obtained by projecting tomographic abnormality information about a second predetermined retinal layer onto the surface along the fundus of the eye to be inspected.

In the first embodiment, the tomographic abnormality map acquisition unit 110 acquires the tomographic abnormality map and layer information of the tomographic abnormality map by the following method. First, the tomographic abnormality map acquisition unit 110 acquires a one-dimensional distribution of the abnormal region by projecting, for each tomographic image for which the tomographic abnormality region has been acquired in Step S34, information on the tomographic abnormality region present in a specific region (second predetermined retinal layer) in a direction perpendicular to the fundus parallel surface. Then, the tomographic abnormality map is acquired by arranging (integrating) the one-dimensional distributions of the abnormal region in the respective tomographic images so as to obtain a two-dimensional map. Here, the user may input the information on the second predetermined retinal layer through use of the input device 11 such as the mouse or the keyboard.

When a configuration in which the tomographic abnormality region of each tomographic image is expressed by a continuous value (abnormality degree) is employed, the tomographic abnormality map may be acquired as the two-dimensional map of the abnormality degree by projecting the abnormality degree in the direction perpendicular to the fundus parallel surface. In this case, the method of projecting the abnormality degree is not limited to maximum intensity projection, and may be any method as long as the method calculates a representative value from a set of abnormality degrees in the projection direction. For example, a 95th percentile value may be used. Further, the tomographic abnormality map may be acquired as a binary two-dimensional map by setting, for the acquired tomographic abnormality map, a region that is equal to or larger than a threshold value set in advance as 1 (abnormal) and a region that is smaller than the threshold value as 0 (normal).

Next, the tomographic abnormality map acquisition unit 110 acquires the above-mentioned specific region (second predetermined retinal layer) as the layer information of the tomographic abnormality map.

Then, the tomographic abnormality map acquisition unit 110 transmits the acquired tomographic abnormality map and layer information of the tomographic abnormality map to the display control unit 104. In this case, in the first embodiment, the second predetermined retinal layer is a specific retinal layer in the tomographic image, but the second predetermined retinal layer is not limited to including the entire range of the specific retinal layer, and may also be any region in the tomographic image. Further, the tomographic abnormality map acquisition unit 110 may acquire information on the entire tomographic image or the entire retinal layer in place of the second predetermined retinal layer.

### Step S36: Acquisition of Shape Abnormality Back-Projection Region

In a shape abnormality back-projection region acquisition step of Step S36, the shape abnormality back-projection region acquisition unit 111 acquires a shape abnormality back-projection region indicating a region corresponding to the shape abnormality map in the tomographic image.

In the first embodiment, the shape abnormality back-projection region acquisition unit 111 acquires the shape abnormality back-projection region by back-projecting the shape abnormality map onto the specific tomographic image, and transmits the shape abnormality back-projection region together with layer information of the shape abnormality back-projection region to the display control unit 104. In this case, the user may input the tomographic image to be selected in the OCT volume data through use of the input device 11 such as the mouse or the keyboard. In the first embodiment, the user inputs (clicks), on the shape abnormality map display window 402 to be described later, the position of the tomographic image desired to be displayed through use of the mouse so as to determine the position. This position of the tomographic image is also used as the position for displaying the tomographic abnormality region.

In the first embodiment, the back projection is performed by the following procedure. First, the position of the tomographic image for which the shape abnormality map is desired to be back-projected is acquired. The user inputs the position of the tomographic image through use of the input device 11 such as the mouse or the keyboard. Next, the abnormal region (one-dimensional information) on the shape abnormality map corresponding to the position of the tomographic image is acquired. Then, the acquired one-dimensional abnormal region is projected onto a layer region indicated by the layer information of the shape abnormality map on the tomographic image so that the shape abnormality back-projection region is acquired.

In the first embodiment, the layer information (first predetermined retinal layer) of the shape map is acquired as the layer information of the shape abnormality back-projection region.

### Step S37: Control of Display of Abnormality Information

In a display control step of Step S37, the display control unit 104 performs control of displaying at least part of the shape abnormality information and at least part of the tomographic abnormality information such that the at least part of the shape abnormality information and the at least part of the tomographic abnormality information are distinguishable from each other.

In the first embodiment, the display control unit 104 generates display information expressing the shape abnormality map, the tomographic abnormality map, the shape abnormality back-projection region, and the tomographic abnormality region in a distinguishable form. Then, the display control unit 104 performs control of displaying, on the display device 12, the generated display information, the acquired layer information, and position information about the tomographic abnormality region serving as a display target.

Now, an example of performing control in which ILM to RPE are set as each of the first predetermined retinal layer and the second predetermined retinal layer, and the shape abnormality information and the tomographic abnormality information obtained for each retinal layer are integrated and displayed is used and described.

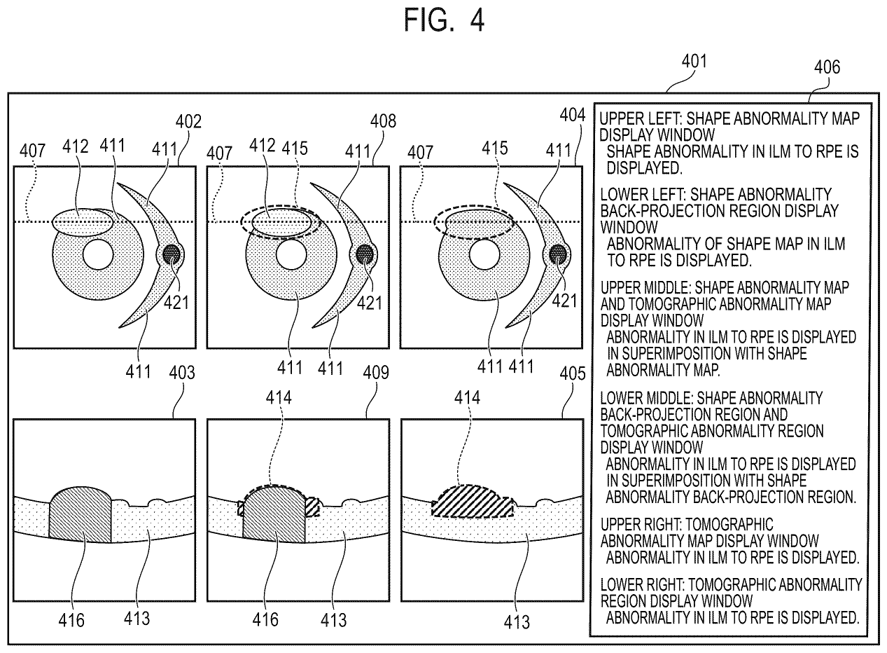

FIG. 4 shows an example of a screen (display screen 401) displaying the display information generated by the display control unit 104 on the display device 12. The display screen 401 includes the shape abnormality map display window 402, a shape abnormality back-projection region display window 403, a tomographic abnormality map display window 404, a tomographic abnormality region display window 405, and a detailed information window 406. The display screen 401 further includes a shape abnormality map and tomographic abnormality map display window 408 and a shape abnormality back-projection region and tomographic abnormality region display window 409. In FIG. 4, a position 407 of the tomographic image indicated by the shape abnormality back-projection region display window 403, the tomographic abnormality region display window 405, and the shape abnormality back-projection region and tomographic abnormality region display window 409 is visualized.

The information displayed in each of the shape abnormality map display window 402, the tomographic abnormality map display window 404, and the shape abnormality map and tomographic abnormality map display window 408 includes the shape map. In this case, for the sake of easy description, the shape map displayed in each of the windows 402, 404, and 408 is an image binarized based on a predetermined threshold value for the layer thickness. That is, each of the windows 402, 404, and 408 includes a shape region 411 in which the thickness of the retinal layer is larger than the predetermined threshold value. A region 421 indicates a region subjected to mask processing of filling the pixels with black pixels for an optic nerve head for which the thickness of the retinal layer cannot be measured.

Further, the information displayed in each of the shape abnormality back-projection region display window 403, the tomographic abnormality region display window 405, and the shape abnormality back-projection region and tomographic abnormality region display window 409 includes the tomographic image at the position (dotted line) 407 (hereinafter also simply referred to as “display tomographic image”). In this case, for the sake of easy description, the display tomographic image displayed in each of the windows 403, 405, and 409 is a binarized image processed so that a difference between a region indicating the retinal layer and a region other than this region becomes clear. That is, each of the windows 403, 405, and 409 includes a retinal layer region 413 in the display tomographic image.

Regarding the position (dotted line) 407, for example, the information processing apparatus 1 may further include a focus position designation unit, and the focus position designation unit may identify the position (dotted line) 407. That is, for example, the information processing apparatus 1 may further include a focus position designation unit for designating a focus position on the shape abnormality map. In addition, the display control unit 104 may be configured to be able to perform control of displaying the tomographic abnormality region and the shape abnormality back-projection region in the tomographic image including the above-mentioned focus position. For example, the user may input information about the focus position to be designated by the focus position designation unit, from the input device 11. Further, the present invention is not limited thereto, and the focus position designation unit may designate the focus position on the shape map or may designate the focus position on the tomographic abnormality map. Further, the display control unit 104 may be configured to be able to perform control of displaying any one of the tomographic abnormality region or the shape abnormality back-projection region in the tomographic image including the focus position.

In the shape abnormality map display window 402, the shape abnormality map acquired in Step S33 and the position (dotted line) 407 of the display tomographic image are displayed in superimposition on the shape map of the first predetermined retinal layer acquired in Step S31. That is, the shape abnormality map display window 402 includes a shape abnormality projection region 412 indicating a region determined as having the shape abnormality in the shape abnormality map. In this case, when a plurality of shape abnormality maps are acquired in Step S33, control may be performed so that the shape maps and the shape abnormality maps are sequentially switched and displayed in response to the operation of the user, or displayed side by side.

In the shape abnormality back-projection region display window 403, a shape abnormality back-projection region 416 acquired in Step S36 is displayed in superimposition on the display tomographic image.

In the shape abnormality back-projection region and tomographic abnormality region display window 409, a tomographic abnormality region 414 acquired in Step S34 and the shape abnormality back-projection region 416 acquired in Step S36 are displayed in superimposition such that those regions are distinguishable from each other on the display tomographic image. The tomographic abnormality region 414 and the shape abnormality back-projection region 416 can be distinguished from each other by displaying each of the regions and a boundary between the regions in an emphasized manner in display modes different from each other.

In this case, the shape abnormality back-projection region 416 is a region obtained by identifying the abnormal region on the shape map and then back-projecting the abnormal region onto the tomographic image, unlike the tomographic abnormality map in which the abnormal region is identified on the tomographic image. Accordingly, a disease that does not appear as an abnormality in the shape map is not reflected in the shape abnormality back-projection region 416. As described above, in some cases, the shape abnormality back-projection region 416 and the tomographic abnormality region 414 may display different regions. Specifically, the following four types exist.
Type 1: a region which is a tomographic abnormality region but is not a shape abnormality back-projection region
Type 2: a region which is both of a tomographic abnormality region and a shape abnormality back-projection region
Type 3: a region which is not a tomographic abnormality region but is a shape abnormality back-projection region
Type 4: a region which is neither a tomographic abnormality region nor a shape abnormality back-projection region

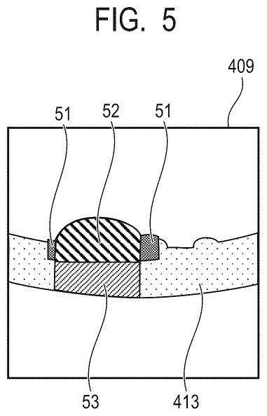

In view of the above, the display control unit 104 can perform control of displaying a region in which the shape abnormality back-projection region 416 and the tomographic abnormality region 414 have a difference, in a further emphasized manner so that the above-mentioned four types can be distinguished from each other. FIG. 5 shows an example in which, in the shape abnormality back-projection region and tomographic abnormality region display window 409 in the display screen 401, a Type-1 region 51, a Type-2 region 52, and a Type-3 region 53 are displayed in a state of being further emphasized from FIG. 4.

Specifically, the Type-1 region 51 (outside of the shape abnormality back-projection region 416 in the tomographic abnormality region 414) has a possibility of a disease that does not appear as an abnormality of thickness in the shape map, and hence is displayed in a display mode 1 (for example, this region is displayed with shading of a color 1). The Type-2 region 52 (region in which the tomographic abnormality region 414 and the shape abnormality back-projection region 416 overlap each other) has a high reliability as an abnormal region, and hence is displayed in a display mode 2 (for example, this region is displayed with shading of a color 2). The Type-3 region 53 (outside of the tomographic abnormality region 414 in the shape abnormality back-projection region 416) has a possibility of segmentation error of the shape map, and hence is displayed in a display mode 3 (for example, this region is displayed with shading of a color 3). In addition, a Type-4 region (region other than the Type-1 region 51, the Type-2 region 52, and the Type-3 region 53, which is outside of the shape abnormality back-projection region 416 and outside of the tomographic abnormality region 414) has a high reliability as a normal region. Accordingly, the Type-4 region is displayed in a display mode 4 (for example, this region is displayed without color). Further, details such as which display mode corresponds to which region may be described in an explanatory note or a pop-up window. In addition, when the regions are displayed in those four display modes, the difference between the shape abnormality back-projection region 416 and the tomographic abnormality region 414 can be displayed in a distinguished manner. The Type-1 region 51 to the Type-4 region may each be switched to be displayed or not displayed in an emphasized manner on each display window in response to the operation of the user. Further, the display mode 1 to the display mode 4 may each perform, in addition to shading of the region with a color, display of only a display mode such as a dotted line that makes a boundary distinguishable, or any other methods may be employed.

In the tomographic abnormality map display window 404, the tomographic abnormality map acquired in Step S35 and the position (dotted line) 407 of the display tomographic image are displayed in superimposition on the shape map of the first predetermined retinal layer acquired in Step S31. That is, the tomographic abnormality map display window 404 includes a tomographic abnormality projection region 415 indicating a region in which the tomographic abnormality region is projected in the tomographic abnormality map. In this case, when a plurality of tomographic abnormality maps are acquired in Step S35, the tomographic images and the tomographic abnormality maps may be sequentially switched and displayed in response to the operation of the user, or may be displayed side by side.

In the tomographic abnormality region display window 405, the tomographic abnormality region 414 acquired in Step S34 is displayed in superimposition on the display tomographic image. In this case, when the tomographic abnormality region has been acquired for each of the B-scan images forming the OCT volume data in Step S34, the display tomographic images (B-scan images) and the tomographic abnormality regions 414 may be sequentially switched and displayed in response to the operation of the user. It is preferred that the display tomographic images to be displayed in the shape abnormality back-projection region display window 403, the shape abnormality back-projection region and tomographic abnormality region display window 409, and the tomographic abnormality region display window 405 be the same B-scan image. Further, in this case, when the B-scan image is switched, it is preferred that the respective display tomographic images be switched in association therewith.

In the shape abnormality map and tomographic abnormality map display window 408, the tomographic abnormality map acquired in Step S35 and the shape abnormality map acquired in Step S33 are displayed in superimposition on the shape map acquired in Step S31 so that a difference between regions is distinguishable. Further, the position (dotted line) 407 of the display tomographic image is displayed in superimposition. The expression “distinguishing the difference between regions” means displaying each of the regions or a boundary therebetween in an emphasized manner in a different display mode.

Further, the shape abnormality map is a map in which the abnormal region is identified based on the shape abnormality information, unlike the tomographic abnormality map obtained by projecting the abnormal region identified on the tomographic image onto the fundus parallel surface. Accordingly, a disease in a state not identified as an abnormality in the shape abnormality information is not reflected on the shape abnormality map. That is, in some cases, the shape abnormality map and the tomographic abnormality map may display different regions. Specifically, the following four types exist.
Type 5: a region indicated as an abnormal region in the tomographic abnormality map but not indicated as an abnormal region in the shape abnormality map
Type 6: a region indicated as an abnormal region in both of the tomographic abnormality map and the shape abnormality map
Type 7: a region not indicated as an abnormal region in the tomographic abnormality map but indicated as an abnormal region in the shape abnormality map
Type 8: a region not indicated as an abnormal region in any of the tomographic abnormality map and the shape abnormality map

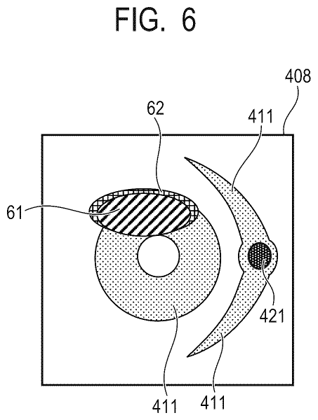

In view of the above, the display control unit 104 can perform control of displaying a region in which the shape abnormality map and the tomographic abnormality map have a difference, in a further emphasized manner so that the above-mentioned four types can be distinguished from each other. FIG. 6 shows an example in which a Type-5 region 62 and a Type-6 region 61 are displayed in the shape abnormality map and tomographic abnormality map display window 408 in the display screen 401.

Specifically, the Type-5 region 62 (region obtained by subtracting the shape abnormality projection region 412 from the tomographic abnormality projection region 415) has a possibility of a disease that does not appear as an abnormality of thickness in the shape map, and hence is displayed in a display mode 1 (for example, this region is displayed with shading of a color 1). The Type-6 region 61 (region in which the tomographic abnormality projection region 415 and the shape abnormality projection region 412 overlap each other) has a high reliability as the abnormal region, and hence is displayed in a display mode 2 (for example, this region is displayed with shading of a color 2). A Type-7 region (no corresponding region in FIG. 6) has a possibility of segmentation error of the shape map, and hence is displayed in a display mode 3 (for example, this region is displayed with shading of a color 3). In addition, a Type-8 region (region of a complementary set of the tomographic abnormality projection region 415) has a high reliability as the normal region, and hence is displayed in a display mode 4 (for example, this region is displayed without color). Further, details such as which display mode corresponds to which region may be described in an explanatory note or a pop-up window. In addition, when the regions are displayed in those four display modes, the difference between the shape abnormality map and the tomographic abnormality map can be displayed in a distinguished manner. In this case, the Type-5 region to the Type-8 region may each be switched to be displayed or not displayed in an emphasized manner on each display window in response to the operation of the user.

In the manner described above, on the display screen 401, the shape abnormality map and the tomographic abnormality map that are related to the two-dimensional map obtained by projecting the abnormal region onto the fundus parallel surface are displayed in an upper part of the display screen 401. That is, the shape abnormality map display window 402, the tomographic abnormality map display window 404, and the shape abnormality map and tomographic abnormality map display window 408 are displayed in the upper part of the display screen 401. Further, the shape abnormality back-projection region 416 and the tomographic abnormality region 414 that are related to the abnormal region in the tomographic image are displayed in a lower part of the display screen 401. That is, the shape abnormality back-projection region display window 403, the tomographic abnormality region display window 405, and the shape abnormality back-projection region and tomographic abnormality region display window 409 are displayed in the lower part of the display screen 401. In this manner, display can be performed so that the images are distinguishable in the respective windows based on a difference between upper and lower sides of the window positions displayed on the display screen 401.

Moreover, on the display screen 401, information related to the shape abnormality is displayed on the left side of the display screen 401, that is, in the shape abnormality map display window 402 and the shape abnormality back-projection region display window 403. Meanwhile, information related to the tomographic abnormality is displayed on the right side of the display screen 401, that is, in the tomographic abnormality map display window 404 and the tomographic abnormality region display window 405. In addition, information indicating both of a shape abnormality and a tomographic abnormality is displayed in the middle of the display screen 401, that is, in the shape abnormality map and tomographic abnormality map display window 408 and the shape abnormality back-projection region and tomographic abnormality region display window 409. In this manner, display can be performed so that the images are distinguishable in the respective windows based on a difference between right and left sides of the window positions displayed on the display screen 401.

In addition, details of those images are displayed in the detailed information window 406.

The “details of the image” refer to the layer information of the plurality of shape abnormality maps, the layer information of the tomographic abnormality region, the position of the tomographic image, and the like, but any other related information may be displayed. For example, it is assumed that the shape abnormality map displays ILM to RPE, and the tomographic abnormality region also has the abnormal region in ILM to RPE. In this case, details such as “ILM to RPE layers are displayed.” can be displayed as the layer information of the shape abnormality map. Further, for example, details such as “There is an abnormality in ILM to RPE layers. The tomographic image displays a tomographic image at positional coordinates of the center.” can be displayed as the layer information of the tomographic abnormality region. Further, regarding the shape map, the fact that there is an abnormality also in other shape abnormality maps (ILM to GCL, ILM to INL) may be displayed.

FIG. 4 shows an example in which, for the sake of easy description, each of the shape map, the tomographic image, the shape abnormality map, and the tomographic abnormality map is binarized and displayed, but the present invention is not limited thereto. For example, the picked-up image may be directly used as the display tomographic image, or the information desired to be displayed may be subjected to each type of processing so as to emphasize the information and displayed. Further, a value corresponding to the shape information on the retinal layer such as the layer thickness or a continuous value of a value indicating the degree of the abnormality may be displayed by a heat map or the like. Further, also the tomographic abnormality region 414 and the shape abnormality back-projection region 416 may display a continuous value of a value indicating the degree by a heat map or the like.

According to the information processing apparatus of the first embodiment, the difference between the abnormal region that is based on the shape abnormality information and the abnormal region detected in the tomographic image can be displayed in a user-friendly manner based on the difference in the displayed window and the difference in the display mode between the respective regions in each of the windows.

In the first embodiment, the order of the steps of Step S31 to Step S36 can be changed as appropriate. That is, it is only required that the tomographic abnormality map be acquired after the tomographic abnormality information is acquired, the shape abnormality map be acquired after the shape abnormality information is acquired, and then the shape abnormality back-projection region be acquired. The detailed step order may be changed as appropriate.

### Modification Example 1 of First Embodiment

In the above-mentioned first embodiment, in Step S37, the display control unit 104 displays the shape abnormality map, the shape abnormality back-projection region, the tomographic abnormality map, and the tomographic abnormality region such that those are distinguishable from each other. However, the embodiment of the present invention is not limited thereto, and the windows may be displayed in any combination as long as a configuration that can display the shape abnormality information and the tomographic abnormality information such that those are distinguishable from each other is employed. For example, there may be employed a configuration in which the display screen 401 displays only the shape abnormality map and tomographic abnormality map display window 408 and/or the shape abnormality back-projection region and tomographic abnormality region display window 409. Further, there may be employed a configuration in which the shape abnormality map and tomographic abnormality map display window 408 and/or the shape abnormality back-projection region and tomographic abnormality region display window 409 is not displayed. Further, a configuration in which the display in the lower part is omitted and a configuration in which the display in the upper part is omitted may be employed. Further, a configuration in which only the detailed information window 406 is displayed may be employed. Further, the windows may be displayed on the display screen 401 in any other combinations, including the shape abnormality map display window 402, the shape abnormality back-projection region display window 403, the tomographic abnormality map display window 404, and the tomographic abnormality region display window 405. Further, for the combination, a specific acquisition unit may be omitted from the hardware configuration of the information processing apparatus.

When only the shape abnormality map and tomographic abnormality map display window 408 is displayed on the display screen 401, a region having abnormality in the shape abnormality map and a region having abnormality in the tomographic abnormality map can be displayed in a distinguished manner.

When only the shape abnormality back-projection region and tomographic abnormality region display window 409 is displayed on the display screen 401, a region having abnormality in the shape abnormality back-projection region and a region having abnormality in the tomographic abnormality region can be displayed in a distinguished manner.

When only the detailed information window 406 is displayed on the display screen 401, for example, the display control unit 104 may perform control of displaying layer information having the abnormality. Specifically, for example, when the layers having the abnormality in the shape abnormality map and the layers having the abnormality in the tomographic abnormality region are ILM to GCL, the display control unit 104 performs control of displaying “There is abnormality in ILM to GCL in both of the shape abnormality map and the tomographic abnormality region.” In this manner, the layers having the abnormality in the shape abnormality map and the layers having the abnormality in the tomographic abnormality region can be displayed in a distinguished manner.

Further, the display control unit 104 may be configured to store the generated display information into the storage unit 105 as an image. Further, the display control unit 104 may be configured to output the generated display information to an external server or the like. In those cases, the control of directly displaying the display information on the display device 12 is not always required to be performed.

### Modification Example 2 of First Embodiment

In the above-mentioned first embodiment, in Step S37, the display control unit 104 displays the shape abnormality map, the shape abnormality back-projection region, the tomographic abnormality map, and the tomographic abnormality region such that those are distinguishable from each other. However, the embodiment of the present invention is not limited thereto. There may be employed a configuration in which, when the layer information displayed in the shape abnormality map and the layer information displayed in the tomographic abnormality map are different from each other, the display control unit 104 performs control of displaying at least one of the pieces of layer information.

That is, the display control unit 104 may be configured to be able to perform, when the first predetermined retinal layer and the second predetermined retinal layer are not the same, control of displaying information on at least one of the first predetermined retinal layer and the second predetermined retinal layer.

Specifically, when the layer information displayed in the shape abnormality map is ILM to GCL and the layer information displayed in the tomographic abnormality map is ILM to INL, the display control unit 104 performs control of displaying at least one or more of the layer information of the shape abnormality map or the layer information of the tomographic abnormality map. In this manner, the layers having the abnormality in the shape abnormality map and the layers having the abnormality in the tomographic abnormality map can be displayed in a discriminable manner. In Modification Example 2, the display of the detailed information window 406 may be omitted.

### Modification Example 3 of First Embodiment

In the above-mentioned first embodiment, in Step S35, the tomographic abnormality map acquisition unit 110 acquires the tomographic abnormality map by projecting the tomographic abnormality region onto the fundus parallel surface through use of the second predetermined retinal layer as a processing range, and transmits the tomographic abnormality map to the display control unit 104. However, the embodiment of the present invention is not limited thereto, and the tomographic abnormality map acquisition unit 110 may be configured to acquire the tomographic abnormality map by setting, as the second predetermined retinal layer, the same retinal layer as the first predetermined retinal layer. That is, the tomographic abnormality map acquisition unit 110 may set the layer for which the tomographic abnormality map is acquired (layer information of the tomographic abnormality map) based on the layer information of the shape map (that is, the first predetermined retinal layer) (that is, the layer for which the projection processing is performed may be limited).

Specifically, for example, when the layer information of the shape map is ILM to GCL, the tomographic abnormality map acquisition unit 110 may set the layer information of the tomographic abnormality map to ILM to GCL, which are the same as the layer information of the shape map. In this manner, the shape abnormality map and the tomographic abnormality map having the same layer information can be displayed in a distinguishable manner.

### Modification Example 4 of First Embodiment

In the above-mentioned first embodiment, in Step S34, the tomographic abnormality region acquisition unit 108 acquires the tomographic abnormality region that may have been caused based on a disease in the tomographic image. However, the embodiment of the present invention is not limited thereto, and the tomographic abnormality region acquisition unit 108 may be configured to acquire the tomographic abnormality region while limiting an analysis target range in the tomographic image, based on the layer information of the shape map (that is, the first predetermined retinal layer). That is, the tomographic abnormality information acquisition unit 103 may be configured to acquire the tomographic abnormality region in the same retinal layer as the first predetermined retinal layer. In this manner, the shape abnormality back-projection region and the tomographic abnormality region having the same layer information can be displayed in a distinguishable manner.

### Modification Example 5 of First Embodiment

In the above-mentioned embodiment, the user inputs and sets the layer information of the shape map (first predetermined retinal layer). However, the embodiment of the present invention is not limited thereto, and the shape map acquisition unit 106 may be configured to set the layer information of the shape map (first predetermined retinal layer) to be the same as the layer information of the tomographic abnormality map (second predetermined retinal layer). That is, the shape abnormality map acquisition unit 109 may be configured to acquire the shape abnormality map by setting, as the first predetermined retinal layer, the same retinal layer as the second predetermined retinal layer. Further, the information processing apparatus 1 may include a determination unit for determining a target retinal layer which is a processing target based on the tomographic abnormality region. In this case, after the processing steps of Step S34 and Step S35, the processing steps of from Step S31 to Step S33 may be carried out. Specifically, when the layer information of the tomographic abnormality map is ILM to INL, the shape map acquisition unit 106 sets the layer information of the shape map to ILM to INL, which are the same as the layer information of the tomographic abnormality map. In this manner, the shape abnormality map and the tomographic abnormality map having the same layer information can be displayed in a distinguishable manner.

### Modification Example 6 of First Embodiment

In the above-mentioned embodiment, the user inputs and sets the layer information of the shape map (first predetermined retinal layer). However, the embodiment of the present invention is not limited thereto, and the shape map acquisition unit 106 may be configured to set the layer information of the shape map (first predetermined retinal layer) to be the same as the layer information of the tomographic abnormality region (information indicating which layer the tomographic abnormality region has been detected in). That is, Modification Example 6 is an example of a case in which, in Modification Example 5, all of the retinal layers in which the tomographic abnormality region has been detected are set to the second predetermined retinal layer. Therefore, in this case, as in Modification Example 5 of the first embodiment, after the processing steps of Step S34 and Step S35, the processing steps of from Step S31 to Step S33 may be carried out. Specifically, when the layer information of the tomographic abnormality region is ILM to INL, the shape map acquisition unit 106 sets the layer information of the shape map to ILM to INL, which are the same as the layer information of the tomographic abnormality region. In this manner, the shape can be analyzed while focusing on the layer in which the abnormality has been detected on the tomographic image. Further, the shape abnormality map and the tomographic abnormality region having the same layer information can be displayed in a distinguishable manner.

### Modification Example 7 of First Embodiment

In the above-mentioned first embodiment, in Step S37, the display control unit 104 designates the position (dotted line) 407 for displaying the tomographic abnormality region on the shape abnormality map display window 402. In addition, the display control unit 104 generates a display image in which the tomographic image at the position (dotted line) 407 and the tomographic abnormality region 414 are superimposed, and displays the display image in the tomographic abnormality region display window 405. In addition, the display control unit 104 generates a display image in which the tomographic image at the same position (dotted line) 407 and the shape abnormality back-projection region 416 are superimposed, and displays the display image in the shape abnormality back-projection region display window 403. Moreover, the display control unit 104 generates a display image in which the tomographic image at the same position (dotted line) 407, the tomographic abnormality region 414, and the shape abnormality back-projection region 416 are superimposed, and displays the display image in the shape abnormality back-projection region and tomographic abnormality region display window 409.

However, the embodiment of the present invention is not limited thereto, and the tomographic images displayed in the shape abnormality back-projection region display window 403 and the tomographic abnormality region display window 405 are not required to be tomographic images at the same position. That is, a position for displaying the shape abnormality back-projection region on the shape abnormality map display window 402 is acquired, and the display image in which the tomographic image at this position and the shape abnormality back-projection region are superimposed is displayed in the shape abnormality back-projection region display window 403. Meanwhile, a position for displaying the tomographic abnormality region on the tomographic abnormality map display window 404 is acquired, and the display image in which the tomographic image at this position and the tomographic abnormality region are superimposed may be displayed in the tomographic abnormality region display window 405. That is, the display control unit 104 may be configured to be able to perform display control such that the position of the tomographic image that displays the shape abnormality back-projection region 416 and the position of the tomographic image that displays the tomographic abnormality region 414 are different from each other. At this time, it is preferred that the shape abnormality back-projection region and tomographic abnormality region display window 409 be prevented from displaying a superimposition image of those images.

### SECOND EMBODIMENT

In a second embodiment, an information processing apparatus for acquiring a shape map based on a curvature of a specific retinal layer is described. Now, regarding details of each configuration of the information processing apparatus according to the second embodiment, parts different from those of the first embodiment are described.

### Shape Map Acquisition Unit 106

The shape map acquisition unit 106 acquires a shape map that is a two-dimensional map obtained by projecting information on a shape of a specific retinal layer (first predetermined retinal layer) visualized in each tomographic image (B-scan image) in the OCT volume data onto the fundus parallel surface for all of the tomographic images. Then, the shape map acquisition unit 106 transmits the acquired shape map to the shape abnormality information acquisition unit 102.

In the second embodiment, the shape map is a curvature at the boundary between the specific retinal layers, and is acquired by the following method. First, segmentation of the retinal layer is executed for each tomographic image in the OCT volume data. Then, out of regions obtained as a result of the segmentation, a one-dimensional distribution of a curvature at a boundary of at least one or more predetermined layers (for example, ILM to RPE) is acquired. Then, the one-dimensional distribution of the curvature (the shape) acquired in each tomographic image is projected onto the fundus parallel surface (mapped on a corresponding line), and the one-dimensional distributions of the curvatures (the shapes) projected in the respective tomographic images are all arranged (integrated) so that a two-dimensional shape map is acquired. A plurality of different shape maps for a plurality of layers (for example, ILM to GCL and ILM to INL) may also be acquired, instead of acquiring one shape map.

How to acquire the shape map is not limited to a method of projecting the information on the shape acquired in each tomographic image described above onto the fundus parallel surface for all of the tomographic images and integrating the results. For example, the shape map may be acquired by, after performing three-dimensional segmentation from the OCT volume data and acquiring the information on the shape, projecting the information on the shape onto the fundus parallel surface.

### Shape Abnormality Information Acquisition Unit 102 and Shape Abnormality Map Acquisition Unit 109

The shape abnormality information acquisition unit 102 acquires the abnormal region (shape abnormality projection region) in the shape map and the layer information thereof to acquire the shape abnormality information. Subsequently, the shape abnormality map acquisition unit 109 generates a shape abnormality map which is a two-dimensional map obtained by imaging the shape abnormality projection region based on the shape abnormality information, and transmits the shape abnormality map and the layer information thereof to the display control unit 104.

In the second embodiment, the shape abnormality map is acquired by the following method. First, through use of the shape map of the eye to be inspected as input, a region having a curvature that exceeds a threshold value set in advance is determined as a region having a shape abnormality. Then, a value of 1 is given to the region having a shape abnormality and a value of 0 is given to other regions so that the shape abnormality map is generated.

In this manner, the shape abnormality map, the shape abnormality back-projection region, the tomographic abnormality map, and the tomographic abnormality region that are based on the curvature of the retinal layer can be displayed such that those are distinguishable from each other.

In the second embodiment, the shape map is acquired based on the curvature at the boundary between the retinal layers, but the shape map may be acquired based on another index.

Any one of the embodiments described above merely indicates an example of implementation for carrying out the present invention, and the technical scope of the present invention is not to be construed in a limiting manner due to those embodiments. That is, the present invention can be carried out in various forms without departing from the technical spirit of the present invention or major features of the present invention. For example, an embodiment in which a configuration of a part of any one of the embodiments is added to another embodiment or an embodiment in which a configuration of a part of any one of the embodiments is substituted by a configuration of a part of another embodiment is also to be understood as an embodiment to which the present invention is applicable.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the present invention, a measure for allowing a relationship between the abnormal region detected from the information on the shape of the retinal layer and the abnormal region detected based on the tomographic image to be easily grasped is provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-203333, filed Nov. 30, 2023, which is hereby incorporated by reference herein in its entirety.

What is claimed is:

1. An information processing apparatus comprising:
a tomographic image acquisition unit configured to acquire a tomographic image of a fundus of an eye to be inspected;
a shape abnormality information acquisition unit configured to acquire, based on the tomographic image, shape abnormality information which is information about a part having an abnormal shape in a retinal layer of the eye to be inspected;
a tomographic abnormality information acquisition unit configured to acquire, based on a tomographic abnormality region which is an abnormal region in the tomographic image, tomographic abnormality information which is information about a part having the tomographic abnormality region in the retinal layer of the eye to be inspected; and
a display control unit configured to perform control of displaying at least part of the shape abnormality information and at least part of the tomographic abnormality information such that the at least part of the shape abnormality information and the at least part of the tomographic abnormality information are distinguishable from each other.

2. The information processing apparatus according to claim 1, further comprising an abnormality map acquisition unit configured to acquire an abnormality map representing at least part of the shape abnormality information and at least part of the tomographic abnormality information on at least one of a surface along the fundus and a surface in a direction intersecting perpendicularly with the fundus,
wherein the display control unit is configured to perform control of displaying the abnormality map.

3. The information processing apparatus according to claim 2,
wherein the abnormality map acquisition unit includes a shape abnormality map acquisition unit and a tomographic abnormality map acquisition unit,
wherein the shape abnormality map acquisition unit is configured to acquire a shape abnormality map which is a map representing the shape abnormality information about a first predetermined retinal layer on the surface along the fundus,
wherein the tomographic abnormality map acquisition unit is configured to acquire a tomographic abnormality map which is a map obtained by projecting the tomographic abnormality information about a second predetermined retinal layer onto the surface along the fundus, and
wherein the display control unit is configured to perform control of displaying the shape abnormality map and the tomographic abnormality map.

4. The information processing apparatus according to claim 2,
wherein the abnormality map acquisition unit includes a shape abnormality map acquisition unit and a shape abnormality back-projection region acquisition unit,
wherein the shape abnormality map acquisition unit is configured to acquire a shape abnormality map which is a map representing the shape abnormality information about a first predetermined retinal layer on the surface along the fundus,
wherein the shape abnormality back-projection region acquisition unit is configured to acquire a shape abnormality back-projection region indicating a region corresponding to the shape abnormality map in the tomographic image, and
wherein the display control unit is configured to perform control of displaying the shape abnormality back-projection region and the tomographic abnormality region.

5. An information processing apparatus comprising:
a tomographic abnormality region acquisition unit configured to acquire a tomographic abnormality region which is an abnormal region in a tomographic image of a fundus of an eye to be inspected;
a determination unit configured to determine a target retinal layer that is a processing target based on the tomographic abnormality region;
a shape abnormality map acquisition unit configured to acquire, based on the tomographic image, a shape abnormality map representing information about a part having an abnormal shape in the target retinal layer on a surface along the fundus; and
a display control unit configured to perform control of displaying the shape abnormality map.

6. The information processing apparatus according to claim 3, wherein the display control unit is configured to perform control of displaying, when the first predetermined retinal layer and the second predetermined retinal layer are not the same, information on at least one of the first predetermined retinal layer and the second predetermined retinal layer.

7. The information processing apparatus according to claim 3, wherein the tomographic abnormality map acquisition unit is configured to acquire the tomographic abnormality map by setting, as the second predetermined retinal layer, the same retinal layer as the first predetermined retinal layer.

8. The information processing apparatus according to claim 3, wherein the tomographic abnormality information acquisition unit is configured to acquire the tomographic abnormality region in the same retinal layer as the first predetermined retinal layer.

9. The information processing apparatus according to claim 4, wherein the tomographic abnormality information acquisition unit is configured to acquire the tomographic abnormality region in the same retinal layer as the first predetermined retinal layer.

10. The information processing apparatus according to claim 3, wherein the shape abnormality map acquisition unit is configured to acquire the shape abnormality map by setting, as the first predetermined retinal layer, the same retinal layer as the second predetermined retinal layer.

11. The information processing apparatus according to claim 4, further comprising a focus position designation unit configured to designate a focus position on the shape abnormality map,
wherein the display control unit is configured to perform control of displaying the tomographic abnormality region and the shape abnormality back-projection region in the tomographic image including the focus position.

12. The information processing apparatus according to claim 4, wherein the display control unit is configured to perform display control such that a position of the tomographic image that displays the shape abnormality back-projection region and a position of the tomographic image that displays the tomographic abnormality region are different from each other.

13. The information processing apparatus according to claim 3,
wherein the shape abnormality map acquisition unit is configured to acquire the shape abnormality map based on a difference between information on a shape of a retinal layer of a healthy eye and information on a shape of a retinal layer acquired from the tomographic image of the fundus of the eye to be inspected, the information on the shape of the retinal layer of the healthy eye being obtained by inputting the information on the shape of the retinal layer acquired from the tomographic image of the fundus of the eye to be inspected to a trained model, and
wherein the trained model is a trained model configured to receive the information on the shape of the retinal layer acquired from the tomographic image of the fundus as input, and output the information on the shape of the retinal layer of the healthy eye for the retinal layer corresponding to the input.

14. The information processing apparatus according to claim 13, wherein the information on the shape of the retinal layer is one of a layer thickness or a curvature.

15. The information processing apparatus according to claim 4,
wherein the shape abnormality map acquisition unit is configured to acquire the shape abnormality map based on a difference between information on a shape of a retinal layer of a healthy eye and information on a shape of a retinal layer acquired from the tomographic image of the fundus of the eye to be inspected, the information on the shape of the retinal layer of the healthy eye being obtained by inputting the information on the shape of the retinal layer acquired from the tomographic image of the fundus of the eye to be inspected to a trained model, and
wherein the trained model is a trained model configured to receive the information on the shape of the retinal layer acquired from the tomographic image of the fundus as input, and output the information on the shape of the retinal layer of the healthy eye for the retinal layer corresponding to the input.

16. The information processing apparatus according to claim 15, wherein the information on the shape of the retinal layer is one of a layer thickness or a curvature.

17. The information processing apparatus according to claim 5,
wherein the shape abnormality map acquisition unit is configured to acquire the shape abnormality map based on a difference between information on a shape of a retinal layer of a healthy eye and information on a shape of a retinal layer acquired from the tomographic image of the fundus of the eye to be inspected, the information on the shape of the retinal layer of the healthy eye being obtained by inputting the information on the shape of the retinal layer acquired from the tomographic image of the fundus of the eye to be inspected to a trained model, and
wherein the trained model is a trained model configured to receive the information on the shape of the retinal layer acquired from the tomographic image of the fundus as input, and output the information on the shape of the retinal layer of the healthy eye for the retinal layer corresponding to the input.

18. The information processing apparatus according to claim 17, wherein the information on the shape of the retinal layer is one of a layer thickness or a curvature.

19. An information processing method comprising:
a tomographic image acquisition step of acquiring a tomographic image of a fundus of an eye to be inspected;
a shape abnormality information acquisition step of acquiring, based on the tomographic image, shape abnormality information which is information about a part having an abnormal shape in a retinal layer of the eye to be inspected;
a tomographic abnormality information acquisition step of acquiring, based on a tomographic abnormality region which is an abnormal region in the tomographic image, tomographic abnormality information which is information about a part having the tomographic abnormality region in the retinal layer of the eye to be inspected; and
a display control unit step of performing control of displaying at least part of the shape abnormality information and at least part of the tomographic abnormality information such that the at least part of the shape abnormality information and the at least part of the tomographic abnormality information are distinguishable from each other.

20. A non-transitory storage medium having stored thereon a program for causing a computer to execute the information processing method of claim 19.
